(19) United States
(12) Patent Application Publication
(10) Pub. No.: US 2025/0177534 A1
(43) Pub. Date: Jun. 5, 2025

(54) THERMOSTABLE UV INACTIVATED VACCINES AND OTHER BIOPHARMACEUTICALS

(71) Applicant: Universal Stabilization Technologies Inc., San Diego, CA (US)

(72) Inventors: Victor Bronshtein, San Diego, CA (US); Min-Xuan Wang, San Diego, CA (US)

(73) Assignee: Universal Stabilization Technologies Inc., San Diego, CA (US)

(21) Appl. No.: 18/840,732

(22) PCT Filed: Feb. 23, 2023
(86) PCT No.: PCT/US2023/013736

§ 371 Date: Aug. 22, 2024

Related U.S. Application Data
(60) Provisional application No. 63/372,226, filed on Feb. 24, 2022.

(51) Int. Cl. A61K 41/17; A61K 39/00; A61K 39/02; A61K 39/07; A61K 39/108; A61K 39/112; A61K 39/12

(52) U.S. Cl. CPC A61K 41/17; A61K 39/0002; A61K 39/0208; A61K 39/0258; A61K 39/0275; A61K 39/0291; A61K 39/07; A61K 39/107; A61K 39/12; A61K 2039/5252

(57) ABSTRACT

This invention describes method for inactivation of microorganisms in thermostable dry formulations at ambient temperatures (AT) using UV light irradiation. According to this method microorganisms are inactivated at ambient temperatures (AT) in dry formulations where the amount of free radicals formed is relatively small and damage of nucleic acids is the main cause for the microorganism's death. The method will allow production of thermostable inactivated vaccines from wild type and live attenuated microorganisms, thermostable inactivated microbiome products, and thermostable sterilized none-live blood components, therapeutic proteins, antibodies and other fragile biopharmaceuticals.

### TECHNICAL FIELD

The claimed invention relates to potent thermostable ultraviolet (UV) inactivated vaccines and thermostable UV sterilized biopharmaceuticals; and more particularly, to such vaccines with improved inactivation without loss of antigenicity.

### BACKGROUND ART

Globalization of the world economy and the modern itinerant way of life increases probability of spreading of influenza, RSV, covid and other infectious disease outbreaks worldwide. Wild type (WT) virus inactivation has been successfully used for accelerated vaccine development. Well known examples are chemically (e.g., formaldehyde or propiolactone) inactivated Salk polio, eastern equine encephalitis vaccine, and tick-born encephalitis vaccines. However, there are cases where chemical activation of certain vaccines (e.g., measles and respiratory syncytial virus (RSV)) leads to an enhanced or atypical course of the disease after subsequent vaccination. Several chemically inactivated vaccine candidates against coronaviruses were found to cause Th2-type immunopathology and induce antibody-dependent enhancement (ADE) of infectivity and eosinophilia. The immunopathological effects of ADE are characterized by enhancement of viral entry and induction of a severe inflammatory response. Recently, studies on patients with severe COVID-19 have found an increased IgG response, which is an indication of possible ADE of SARS-CoV-2 infection. These observations could be used to question one's approach for development of a vaccine against COVID-19 and it's variants, RSV and some other viruses using chemical inactivation. It was previously demonstrated that electron beam inactivation of RSV generates a potent vaccine with no ADE symptoms in mice, which indicates that the ADE induction could have been because the vaccines had been produced by chemical inactivation, which damaged viral antigenic determinants. Alternatively, ultraviolet, gamma-ray and electron beam irradiation have the ability to inactivate pathogens by damaging nucleic acids rather than proteins, leaving key epitopes undamaged.

Ability of Electron beam (EB), Gamma, X-rays, Ultraviolet (UV) light and other radiation types to inactivate (sterilize) biological specimens is well known. Like other types of irradiation UV light is an ionizing radiation initiating advanced oxidation processes (AOPs). UV-based AOPs have shown the potential for water and wastewater disinfection and pollutant degradation. This combined treatment has caused a synergistic effect on one hand by the direct exposure to UV light, and on the other hand, by the generation of free radicals such as (H·) and hydroxyl radical (OH·) from the UV light. The generation of free radicals due to UV light exposure is considered as the vital element of AOPs. These free radicals have resulted in cell membrane disruption, damaged RNA, DNA and other biological polymers (protein), and eventual death of microorganisms.

UV irradiation of aqueous viral suspensions is conventionally known for development of inactivated vaccines. U.S. Pat. No. 4,693,981, by Wiesehahn et al. and U.S. Pat. No. 9,005,633B2 by Kochel et al. disclosed an improved methods for preparing UV inactivated viral vaccine without substantially degrading its antigenic characteristics that must be carried out in absence of oxygen or oxygenized species to preserve the antigenic characteristics of the virus that could be damaged by free radicals. The absence of oxygen and oxygenized species were maintained by removing the oxygenized species from the inactivation medium prior to irradiation through flushing with non-oxidizing gas and adding oxygen scavengers to the medium. This makes the UV inactivation procedure of the viral suspension very complex. In addition, after the inactivation liquid vaccines still were not stable at ambient temperatures.

The present invention relates to inactivated or sterilized thermostable vaccines and other biopharmaceuticals. In part, the invention relates to a method for preparing inactivated vaccines from wild type (WT) and attenuated microorganisms stabilized at ambient temperatures (AT) by immobilization in anhydrous carbohydrate glasses. More specifically, this invention describes method for inactivation of microorganisms in thermostable dry formulations at ambient temperatures (AT) using Ultraviolet (UV) light irradiation. The methods described and claimed herein are differentiated at least because the irradiation is applied to after the materials have been thermostabilized by drying. This eliminates damaging effects of free radicals that are formed during irradiation of a product in a liquid state. UV irradiation in the dry state has been found to inactivate the PBV vaccine composition through virus nucleic acid damage without affecting virus surface structures immobilized in the glass environment, thus preserving integrity of epitopes, and providing an accurate template for vaccine production.

### SUMMARY OF INVENTION

### Technical Problem

There is an urgent need in platform technologies for accelerated development of safe and potent vaccines against emerging and seasonal infectious disease threats. There is also a need for vaccine enhancement strategies and formulation technologies that would allow reduced number of immunizations, increased ease of administration (i.e., self-administration), increased product stability to minimize cold chain requirements, and enhanced cost-effectiveness of vaccine manufacturing.

UV irradiation of aqueous viral suspensions is known to generate free radicals known to degrade antigenic characteristics of a virus and lowering integrity of epitopes. Present solutions in the prior art to mitigate this issue involve the inactivation process to be complex and does not provide stability at ambient temperatures.

### Solution to Problem

The present invention relates to inactivated or sterilized thermostable vaccines and other biopharmaceuticals. In part, the invention relates to a method for preparing inactivated vaccines from wild type (WT) and attenuated microorganisms stabilized at ambient temperatures (AT) by immobilization in anhydrous carbohydrate glasses. More specifically, this invention describes method for inactivation of microorganisms in thermostable dry formulations at ambient temperatures (AT) using Ultraviolet (UV) light irradiation. The methods described and claimed herein are differentiated at least because the irradiation is applied to after the materials have been thermostabilized by drying.

In one aspect, a method is provided for producing a highly antigenic dry thermostable UV inactivated vaccines produced from live wild type (WT) and/or attenuated microorganisms.

In another aspect, a method is provided for producing thermostable UV sterilized or biopharmaceuticals including blood components, stem and other cell derived products like exosomes, cytokines, antibodies, and therapeutic proteins, postbiotics and inactivated microbiome microorganisms.

Various embodiments of these aspects are disclosed and claimed herein.

### Advantageous Effects of Invention

Applying radiation after the materials have been thermostabilized by drying eliminates damaging effects of free radicals that are formed during irradiation of a product in a liquid state. UV irradiation in the dry state has been found to inactivate the PBV vaccine composition through virus nucleic acid damage without affecting virus surface structures immobilized in the glass environment, thus preserving integrity of epitopes, and providing an accurate template for vaccine production.

### BRIEF DESCRIPTION OF THE DRAWINGS

Other features, combinations, and embodiments will be appreciated by one having the ordinary level of skill in the art of antennas and accessories upon a thorough review of the following details and descriptions, particularly when reviewed in conjunction with the drawings, wherein:

FIG. 1 shows a graph of light transmission curve for Wheaton serum vials;

FIG. 2 shows graph of H3N2 LAIV viral activity after UV exposure inactivation;

FIG. 3 shows a graph of H3N2 LAIV viral activity after electron beam inactivation; and

FIG. 4 shows a graph of ultraviolet inactivation of PBV YF-17D.

### DETAILED DESCRIPTION

For purposes of explanation and not limitation, details and descriptions of certain preferred embodiments are hereinafter provided such that one having ordinary skill in the art may be enabled to make and use the invention. These details and descriptions are representative only of certain preferred embodiments, however, a myriad of other embodiments which will not be expressly described will be readily understood by one having skill in the art upon a thorough review of the instant disclosure. Accordingly, any reviewer of the instant disclosure should interpret the scope of the invention only by the claims, as such scope is not intended to be limited by the embodiments described and illustrated herein.

For purposes herein, the terms “microorganism” and “microbe” are intended to be interchangeable, and to broadly include viruses (virions), bacteria (bacterium), vibrio, fungi and/or yeast. To this end, the microorganism selected for use in the described methods can be any known microorganism for which vaccine, therapeutic or other use is sought, as the methods apply to all microorganisms. However, given the current pandemics, the disclosure is tailored toward viruses without intent to limit the scope of the claimed invention.

The term “thermostable” means having the activity decrease less than 0.5 logs after: (i) 1 year of storage at room temperature, (ii) 6 months of storage at 37° C., and (iii) after 2 month at 40° C.

The term “psoralens” includes derivatives thereof.

The term “sterilization” and its complementary words including “sterilized” means any process, physical or chemical, that destroys microorganism life including but not limited to bacterium, viruses, and fungus. Sterilization includes the inactivation of vaccines.

The term “UVA” means ultraviolet radiation having a wavelength between 315 nm and 400 nm as defined by the WHO.

The term “UVB” means ultraviolet radiation having a wavelength between 280 nm and 315 nm as defined by the WHO.

Unless explicitly defined herein, terms are to be construed in accordance with the plain and ordinary meaning as would be appreciated by one having skill in the art.

### General Description of Embodiments

In one aspect, a method of producing a dry thermostable sterilized biopharmaceuticals from a suspension of pathogenic microorganisms is disclosed. The method comprises stabilizing the suspension comprising pathogenic microorganisms at ambient temperatures by immobilizing the microorganisms in a glassy matrix with a glass transition temperature greater than an ambient temperature at which the material will be stored; and subsequently exposing the stabilized microorganisms to ultraviolet radiation at ambient temperatures having a dose between and inclusive of 0.2 J/cm² and 10 J/cm² to decrease viability of the microorganisms.

In some embodiments, the protective glassy matrix may comprise two or more glass forming protective molecules comprising carbohydrates, amino acids, silica, derivatives thereof, polymers, or a combination thereof.

In some embodiments, the glass forming protective molecules may comprise sucrose, trehalose, isomalt and other non-reducing polysaccharides, methylglucoside and other non-reducing derivatives of monosaccharides, glycerol, sorbitol, mannitol, erythritol, other sugar alcohols, or a combination thereof.

In some embodiments, the protective glassy matrix may be produced by drying and subsequent cooling of a preservation mixture comprising the microorganisms and preservation solutions comprising the protective molecules.

In some embodiments, the dose ultraviolet radiation may comprise between and inclusive of 1 J/cm² and 10 J/cm².

In some embodiments, the biopharmaceutical may comprise blood components, stem cells, exosomes, cytokines, antibodies, therapeutic proteins, postbiotics, inactivated microorganism, or a combination thereof.

In some embodiments, the ultraviolet radiation may comprise a wavelength between and inclusive of 280 nm and 315 nm.

In some embodiments, the ultraviolet radiation may comprise a wavelength between and inclusive of 315 nm and 400 nm.

In some embodiments, the method may further comprise adding psoralens to the suspension of pathogenic microorganisms or to a preservation solution prior to immobilizing.

In some embodiments, said psoralen may be selected from the group consisting of 4′-Aminomethyltrioxalen hydrochloride (AMT), 8-Methoxypsoralen (8-MOP), 4,5′,8-Trimethylpsoralen (TMP), or a combination thereof.

In some embodiments, said psoralen may be added at a concentration between and inclusive of 1-200 μg/ml of the preservation solution.

In some embodiments, the concentration may be between and inclusive of 5-25 g/ml.

In some embodiments, said psoralen may be added to a medium in which the biopharmaceutical is grown.

In some embodiments, the preservation mixture may comprise one part by weight monosaccharide derivatives and/or sugar alcohols and at least two parts by weight non-reducing disaccharides.

In some embodiments, said non-reducing disaccharides may comprise sucrose, trehalose, isomalt, or a combination thereof.

In some embodiments, said monosaccharide derivatives may be selected from the group consisting of: methylglucoside, and 2-Deoxy-d-glucose.

In some embodiments, said sugar alcohols may be selected from the group consisting of: glycerol, sorbitol, mannitol, and erythritol.

In some embodiments, the dry thermostable sterilized biopharmaceutical may be stored at a temperature of 40° C., and the glass transition temperature is greater than or equal to 41° C.

In some embodiments, the pathogenic microorganisms may comprise live virions.

In some embodiments, the live virions may be selected from the group consisting of: coronavirae, influenza, rabies, measles, rubella, yellow fever, smallpox, respiratory syncytial, herpes, aids and Arboviruses including dengue-1, dengue-2, dengue-3, dengue-4, or a combination thereof.

In some embodiments, the pathogenic microorganisms may comprise bacteria, fungi, vibrio, yeast, or a combination thereof.

In some embodiments, the pathogenic microorganisms may comprise anthrax, listeria shigella, salmonella, E. coli, Yersinia pestis, or cholera.

In some embodiments, the dry thermostable sterilized biopharmaceutical may comprise an inactivated vaccine.

In some embodiments, the method may further comprise: isolating nucleic acid aptamers specific to the inactivated vaccine.

In some embodiments, said isolating may comprise contacting particles of the inactivated or killed vaccine with the nucleic acid aptamers and filtering retentate, wherein the retentate comprises complexes of particles and the nucleic acid aptamers.

In some embodiments, the method may further comprise selecting a plurality of therapeutic aptamer candidates from the retentate.

In some embodiments, the method may further comprise sequencing the plurality of therapeutic aptamer candidates.

In some embodiments, the method may further comprise synthesizing and purifying at least one of the plurality of therapeutic aptamer candidates to form a therapeutic composition.

In some embodiments, the method may further comprise confirming specific binding of the therapeutic composition to the inactivated vaccine wherein said binding is performed in human serum.

In some embodiments, the method may further comprise identifying non-neutralizing aptamers of the plurality of therapeutic aptamer candidates.

In some embodiments, the method may further comprise combining the non-neutralizing aptamers with the inactivated vaccine to form an enhanced vaccine preparation.

In some embodiments, the dose of ultraviolet radiation comprises between and inclusive of 1 J/cm² and 10 J/cm².

In some embodiments, the ultraviolet radiation may comprise a wavelength between and inclusive of 290 nm and 310 nm.

In another aspect, a method of producing a dry thermostable sterilized biopharmaceutical using Preservation by Vaporization (PBV) is disclosed. The method comprises combining a microorganism suspension and a preservation solution to form a preservation mixture, the microorganism suspension comprising live virions or cellular microorganisms, the preservation solution comprising amino acids, one or more non-reducing disaccharides, and one or more monosaccharide derivatives and/or sugar alcohols; drying the microorganism suspension by vaporization under vacuum to form a mechanically-stable glassy foam, said mechanically-stable glassy foam comprising: less than five percent residual water content, and a glass transition temperature greater than an ambient temperature at which the material will be stored, wherein the live virions or cellular microorganisms are immobilized in the mechanically-stable glassy foam; and exposing the mechanically-stable glassy foam to an ultraviolet radiation having a dose between and inclusive of 0.2 J/cm² and 10 J/cm² to sterilize the biopharmaceutical.

In some embodiments, the microorganism suspension may be preserved inside one or more serum vials.

In some embodiments, the preservation solution may comprise one part by weight monosaccharide derivatives and/or sugar alcohols and at least two parts by weight non-reducing disaccharides.

In some embodiments, said non-reducing disaccharides may comprise sucrose, trehalose, isomalt, or a combination thereof.

In some embodiments said monosaccharide derivatives may be selected from the group consisting of: methylglucoside, and 2-Deoxy-d-glucose.

In some embodiments, said sugar alcohols may be selected from the group consisting of: glycerol, sorbitol, mannitol, and erythritol.

In some embodiments, the dry thermostable sterilized biopharmaceutical may be stored at a temperature of 40° C., and the glass transition temperature is greater than or equal to 41° C.

In some embodiments, the ultraviolet radiation may comprise a wavelength between and inclusive of 280 nm and 400 nm.

In some embodiments, the live virions may be selected from the group consisting of: coronavirae, influenza, rabies, measles, rubella, yellow fever, smallpox, respiratory syncytial, herpes, aids and Arboviruses including dengue-1, dengue-2, dengue-3, dengue-4.

In some embodiments, the cellular microorganisms may comprise bacteria, fungi, vibrio, yeast, or a combination thereof.

In some embodiments, the cellular microorganisms may comprise anthrax, listeria shigella, salmonella, E. coli, Yersinia pestis, or cholera.

In some embodiments, the sterilized biopharmaceutical may further comprise an inactivated vaccine.

In some embodiments, the method further comprises: isolating nucleic acid aptamers specific to the inactivated vaccine.

In some embodiments, said isolating may comprise contacting particles of the inactivated vaccine with the nucleic acid aptamers and filtering retentate, wherein the retentate comprises complexes of particles and the nucleic acid aptamers.

In some embodiments, the method may further comprise selecting a plurality of therapeutic aptamer candidates from the retentate.

In some embodiments, the method may further comprise sequencing the plurality of therapeutic aptamer candidates.

In some embodiments, the method may further comprise synthesizing and purifying at least one of the plurality of therapeutic aptamer candidates to form a therapeutic composition.

In some embodiments, the method may further comprise confirming specific binding of the therapeutic composition to the inactivated vaccine, wherein said binding is performed in human serum.

In some embodiments, the method may further comprise identifying non-neutralizing aptamers of the plurality of therapeutic aptamer candidates.

In some embodiments, the method may further comprise combining the non-neutralizing aptamers with the inactivated vaccine to form an enhanced vaccine preparation.

In some embodiments, the dose of ultraviolet radiation may comprise between and inclusive of 1 J/cm² and 10 J/cm².

In some embodiments, exposing the mechanically-stable glassy foam to the ultraviolet radiation may occur at ambient temperatures.

In some embodiments, drying may be performed using foam drying comprising a primary drying by vaporization including a process of boiling.

In some embodiments, the ultraviolet radiation may comprise a wavelength between and inclusive of 290 nm and 310 nm.

In some embodiments, the dose is greater than 1 J/cm².

In some embodiments, the method may further comprise adding psoralens to the microorganism suspension prior to drying.

In some embodiments, said psoralen may be selected from the group consisting of 4′-Aminomethyltrioxalen hydrochloride (AMT), 8-Methoxypsoralen (8-MOP), 4,5′,8-Trimethylpsoralen (TMP), or a combination thereof.

In some embodiments, said psoralen may be added at a concentration between and inclusive of 1-200 μg/ml.

In some embodiments, the concentration may be between and inclusive of 5-25 g/ml.

In some embodiments, said psoralen may be added to a medium in which the biopharmaceutical is grown.

In some embodiments, the method may further comprise adding psoralens to the preservation solutions prior to drying.

For purposes herein, the terms “microorganism” and “microbe” are intended to be interchangeable, and to broadly include viruses (virions), bacteria (bacterium), vibrio, fungi and/or yeast. To this end, the microorganism selected for use in the described methods can be any known microorganism for which vaccine, therapeutic or other use is sought, as the methods apply to all microorganisms. However, given the current pandemics, the disclosure is tailored toward viruses without intent to limit the scope of the claimed invention. More specifically the method can include any live virions including but not limited to: coronavirae, influenza, rabies, measles, rubella, yellow fever, smallpox, respiratory syncytial (RSV), herpes, dengue or aids. The method can include wherein the cellular microorganisms comprise anthrax, listeria shigella, salmonella, E. coli, Yersinia pestis, or cholera.

Now, in accordance with various embodiments, a method of producing a dry thermostable inactivated vaccine is disclosed, the method is comprising of three steps: (i) combining a suspension comprising one or more microorganisms and a preservation solution (PS) comprising glass forming protective ingredients to form a vaccine preservation mixture (PM). (ii) drying the vaccine PM to less than five percent residual water content needed to transform the PM into a glass with the glass transition temperature Tg above 25° C. to immobilize/preserve the microorganisms in the glass state at ambient temperatures (ATs); and (iii) exposing the microorganisms immobilized in the glass to UV radiation dose needed to produce an inactivated or killed vaccines. The method can include wherein the maximum storage temperature is 40° C., and the glass transition temperature is greater than or equal to 41° C.

The method can include wherein will consider the vaccine as thermostable if it's activity decreases less than 0.5 logs after: (i) 1 year of storage at room temperature, (ii) 6 months of storage at 37° C., and (iii) after 2 months at 40° C.

The list of protective ingredients includes, but is not limited to amino acids, silica, Polyvinylpyrrolidone, hydroxy ethyl starch or other polymeric protectants, one or more non-reducing disaccharide or polysaccharide, and one or more not reducing monosaccharide derivatives and/or sugar alcohols. Non-reducing disaccharides could be selected from the group consisting of: sucrose, trehalose, and isomalt. Monosaccharide derivatives could be selected from the group consisting of: methylglucoside, and 2-Deoxy-d-glucose. Sugar alcohols could be selected from the group consisting of: glycerol, sorbitol, mannitol, and erythritol.

The method can include wherein the cellular microorganisms comprise wild type or microbiome derived bacteria, fungi, vibrio, or yeast.

The method can be applied to any non-live biopharmaceuticals including but not limited to: blood components, stem and other cell derived products like exosomes, cytokines, antibodies, and therapeutic proteins; postbiom products.

The disclosure, in its various aspects and embodiments, suggests (i) thermostabilization of wild type infectious viruses, including without limitation influenza, RSV, Yellow fever, dengue, and SARS-CoV-2 and its variants, by immobilizing the microorganisms in a dry glass matrix with glass transition temperature Tg greater than a maximum temperature at which the product will be stored, then (ii) subsequently exposing the stabilized microorganisms to a UV radiation dose between 0.2 and 10 J/sm² at ambient temperatures to decrease the survival of the microorganisms more than a million times and to produce an inactivated or killed but potent products. Additionally, to ensure both effective thermostabilization of vaccines and other fragile biopharmaceuticals as well as scalability of this method, it is preferable to use for drying Preservation by Vaporization (PBV) process described in U.S. Pat. Nos. 9,469,835 and 11,400,051.

PBV is an industrial scale vacuum drying process that can be executed using conventional lyophilizers without any modification. PBV primary drying is performed by boiling from a partially frozen (slush) state instead of by sublimation from a completely frozen solid state, which is responsible for the instability of Freeze-dried (F-D) vaccines at high ambient temperatures ATs. PBV is also several times quicker and cheaper than F-D.

PBV is a better alternative to Freeze-drying (F-D). It has been found that the activity of most freeze-dried live attenuated vaccines (LAVs) decreases more than 1000 times during 6 months at 37° C. Many LAVs preserved using PBV are stable for years at RT and for at least 6 months at 37° C. PBV has been used to formulate thermostable MVA (Smallpox LAV), YF-17D (Yellow fever LAV), Measles and Rubella LAVs, Influenza H3N2 LAV, ERA-333 (Rabies LAV), and other LAVs. It has also been demonstrated that PBV LAVs can be micronized using jet or ball milling for mucosal delivery with no or minimal viral activity loss.

Harnessing thermostable vaccines for utilization of intranasal and other immune surfaces opens unprecedented opportunities in vaccination. In recent studies supported by NIH and CDC, it was demonstrated that effective intranasal vaccination of ferrets with PBV H3N2 LAV and monkeys with PBV Measles and Rubella; buccal vaccination of piglets with PBV Rotavirus LAVs; and intestinal vaccination of foxes with PBV RRA 333 Rabies LAV. It was found that PBV vaccines delivered to mucosal surfaces in dry powder format elicit both protective systemic and mucosal immune responses needed to stop the spread of many diseases. Preliminary studies also indicate that radiation inactivated thermostable vaccines will produce similar immunogenicity at higher numbers of viral particle per dose.

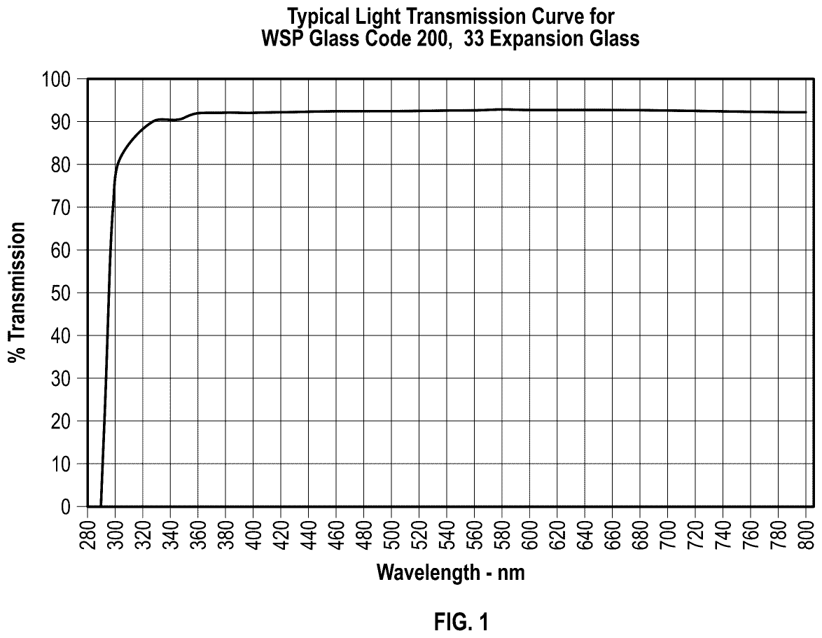

The UV inactivation methods disclosed and claimed herein could be applied to microorganisms which are dried in bulk format (in open trays) or in serum vials. The UV light is categorized into three based on its wavelength as UVA (315 to 400 nm), UVB (280 to 315 nm), and UVC (100 to 280 nm). The wavelength range of 200 to 280 nm for UVC is considered as the germicidal range due to its strong direct damage to the nucleic acids of microorganisms. Nucleic acids are damaged also could be damaged by UVB and UVA, but with lower efficiency than by UVC radiation because the efficiency quickly decreases with increasing wavelength of the radiation. All UVA, UVB and UVC radiation could be used to inactivate products dried in the open trays. However, only UVA and UVB could be used to inactivate microorganisms dried in serum vials because the UVC does not permeate inside glass vials (see FIG. 1). FIG. 1 shows the light transmission curve for the Wheaton serum vials.

Inactivation efficacy of UVA is very low. For example, decrease of viral survival was not detected even after very high (above 1 J/cm²) irradiation dose of UVA at 365 nm wavelength. The inactivation efficiency of UVA can be achieved using psoralens or psoralen derivatives [U.S. Pat. No. 4,693,981, by Wiesehahn et al. and U.S. Pat. No. 9,005,633B2 by Kochel et al. (Kochel et al., 2015.] Good safety record and photo-crosslinking property of psoralens lend it to possible use in inactivating viruses and other microorganisms.

Psoralen (also called psoralene) is the parent compound in a family of natural products known as furocoumarins. Psoralens are photoreactive compounds that are freely permeable in phospholipid membranes and intercalate between double-stranded nucleic acids. Following exposure to long wave ultraviolet radiation (UVA) the intercalated psoralen covalently crosslinks complementary pyrimidine residues, leading to viral inactivation through inhibition of genome replication. Psoralen interaction with viral nucleic acids leaves immunogenic surface epitopes intact, raising the possibility that a psoralen-inactivated virus may serve as a vaccine candidate. The photo-crosslinking property of psoralens has been exploited to inactivate microorganisms in the blood supply for treatment of skin disorders, to inactivate viral pathogens prior to organ transplantation, and for inactivation of viruses for potential vaccines.

Psoralen-inactivated viruses should, in theory, retain their three-dimensional structure, permitting the development of antibodies to multiple epitopes that may participate in immunity. Psoralens do not appear to interact with proteins. Additionally, they only induce cross-linking of pyrimidines following UV exposure. This feature of psoralens has made them attractive in transfusion medicine for pathogen inactivation, wherein they damage the nucleic acid of pathogenic contaminants without disrupting the donor-derived erythrocytes, platelets, and coagulation factors themselves.

Psoralens also have a good safety record in humans. There is increasing experience with the use of psoralens in blood banking, particularly the use of amotosalen as an alternative to traditional leukoreduction methods for the prevention of CMV transmission. The oral and topical use of psoralens in the treatment of psoriasis has been associated with photosensitivity, contact dermatitis, and DNA damage in histologic specimens from treated tissue. These adverse reactions are due in part to the direct exposure of human skin to UVA and psoralen, and do not seem likely to be of concern following vaccine preparation, assuming adequate purification of the inactivated virus preparation.

Psoralen is known to be effective in inactivating viruses by UVA in liquid suspensions the optimal inactivation conditions for such inactivation of microorganisms stabilized at AT in anhydrous state, including the length of UVA exposure, the UV doses, the concentration and selection of psoralen were not investigated. U.S. Pat. Nos. 4,124,598 and 4,196,281 to Hearst et al. suggest the use of psoralen derivatives to inactivate RNA viruses but include no discussion of the suitability of using the inactivated viruses for vaccines. U.S. Pat. No. 4,169,204 to Hearst et al. suggest that psoralens may provide a mean for inactivating viruses for the purpose of vaccine production but presents no experimental support for this proposition. European patent application 0 066 886 by Kronenberg teaches the use of psoralen inactivated cells, such as virus-infected mammalian cells, for use as immunological reagents and vaccines.

Our invention suggests stabilizing microorganisms and other biopharmaceuticals at AT by drying first and after that inactivating them by applying UV. We found that the UVA can inactivate the viruses immobilized in carbohydrate glasses similar to that in the liquid state if psoralens were included (added to) viruses or preservation solutions before drying.

According to Kochel et al., [2015] psoralens may be used in the inactivation process include psoralen and substituted psoralens, in which the substituent may be alkyl, particularly having from one to three carbon atoms, e.g., methyl; alkoxy, particularly having from one to three carbon atoms, e.g., methoxy; and substituted alkyl having from one to six, more usually from one to three carbon atoms and from one to two heteroatoms, which may be oxy, particularly hydroxy or alkoxy having from one to three carbon atoms, e.g., hydroxy methyl and methoxy methyl, or amino, including mono- and dialkyl amino or aminoalkyl, having a total of from zero to six carbon atoms, e.g., aminomethyl. There will be from 1 to 5, usually from 2 to 4 substituent, which will normally be at the 4,5,8,4′ and 5′ positions, particularly at the 4′ position. Illustrative compounds include 5-methoxypsoralen; 8-methoxypsoralen (8-MOP); 4,5′,8-trimethylpsoralen (TMP); 4′-hydroxymethyl-4,5′,8-trimethylpsoralen (HMT); 4′-aminomethyl-4,5′,8-trime-thylpsoralen (AMT); 4-methylpsoralen; 4,4′-dimethylpsoralen; 4,5′-dimethylpsoralen; 4′,8-dimethylpsoralen; and 4′-methoxymethyl-4,5′,8-trimethylpsoralen. Of particular interest are AMT4,5′, TMP and 8-MOP. Different psoralens may be used individually or in combination in the inactivation process. Depending on concentration of microorganisms the psoralens may present in amounts ranging from 1-200 μg/ml, preferably from about 5-25 μg/ml.

In carrying out embodiments of the invention, the psoralen(s) should be added to the preservation mixtures before drying. Psoralens could be included in preservation solutions or combined with viral suspensions. In some cases it may be desirable to introduce psoralens to the virus by addition to a cell culture medium in which the virus is grown. The dose of UVA irradiation will vary depending upon the light intensity, the concentration of the psoralen, the concentration of the virus and the way the irradiation is applied to the dry preserved microorganisms.

These and other embodiments are conceived and supported in accordance with the following experimental examples, wherein:

### Example 1: Thermostabilization of Live Attenuated Influenza Vaccine (LAIV)

PBV was applied to stabilize H3N2 LAIV. Before drying the vaccine was mixed 1:1 with a preservation solution (PS) to form a preservation mixture (PM). Preservation solution comprised: 30% sucrose and 5% mannitol. PM was aliquoted in 5 ml serum vials, 0.5 ml of PM per vial (Wheaton borosilicate glass serum vials #223685). The vials were placed in a Virtis Genesis freeze-drier for stabilization using Preservation by Vaporization (PBV) (see U.S. Pat. No. 946983582) technology. The PBV primary drying was performed by boiling from a partially frozen state, transforming the PM into a glassy foam. The protocol also comprised 20 hours of secondary drying at 45° C. to ensure stability of PBV LAIV at ambient temperatures (AT). At the end of secondary drying, the vials were closed with rubber stoppers and sealed under vacuum. Two batches of PBV-preserved LAIV were produced: the first was used in long-term stability studies to evaluate thermostability of the PBV vaccine. The second was produced following identical protocols and used in inactivation studies. Viral activity was tested after PBV, AT storage (see Table 1 below), and after irradiation. Titers were measured following the TCID50 assay protocol: TCID50 assay is performed by infecting monolayer MOCK-London cells with a series of 1:10 viral dilutions in EMEM media with TPCK-Trypsin (2.5 μg/ml). Cells were kept in a 5% CO2 incubator at 33° C. for 5 days. Endpoint was determined via visual determination of CPE and confirmed by hemagglutination development with 0.5% turkey erythrocytes (tRBC). The tissue culture infectious dose 50% (TCID50) titer was calculated using the Muench and Reed method.

TABLE 1
Stability of PBV-preserved LAIV after 9 months:

| Storage time | Log loss from storage | Titer (TCID50/mL) |
|---|---|---|
| 0 months | 0 | 9.67E+06 |
| 1 months | −0.10 | 7.61E+06 |
| 3 months | −0.55 | 2.72E+06 |
| 9 months | −0.50 | 3.07E+06 |

### Example 2. Inactivation of PBV-Preserved LAIV Using Ultraviolet Irradiation

UV inactivation of the dry vaccine formulation was performed using a midrange UVP CL-1 ODOM Ultraviolet Crosslinker producing UV light primary at wavelength 302 nm. The sealed serum vials containing PBV-preserved LAIV were subjected to a range of UV radiation doses (0.01-1.8 J/cm² of the irradiated area) of ultraviolet irradiation to inactivate the live attenuated virus. Samples were subsequently tested for viral infectivity activity and antigenicity after inactivation to evaluate the effect of irradiation dose on the preserved vaccine. The antigenicity was measured via a standard hemagglutination inhibition (HI) assay using the standardized protocol developed at CDC (protocol LP-064, R-Od (Effective Jul. 11, 2011). HI assay characterizes ability of vaccine epitopes to bind blocking antibodies within hyper-immune ferret serum and agglutinate turkey erythrocytes (tRBC). HI titers are presented using geometric mean of the data, as suggested in literature [Roberts J. Charting influenza titers. J Swine Health Prod. 2002; 10(1):39-40]. In brief, vaccine samples (diluted to 8 HAU/50 ul) were added to serially diluted A/Tx ferret antisera and incubated at RT for 30 minutes. Turkey erythrocytes (0.5%) were added to all samples and incubated 30 minutes at RT. Plates were tilted ˜60 degrees and results recorded. Positive inhibition was recorded as the reciprocal of the last dilution resulting in lack of complete agglutination of RBC. Activity of PBV-preserved LAIV after UV Irradiation:

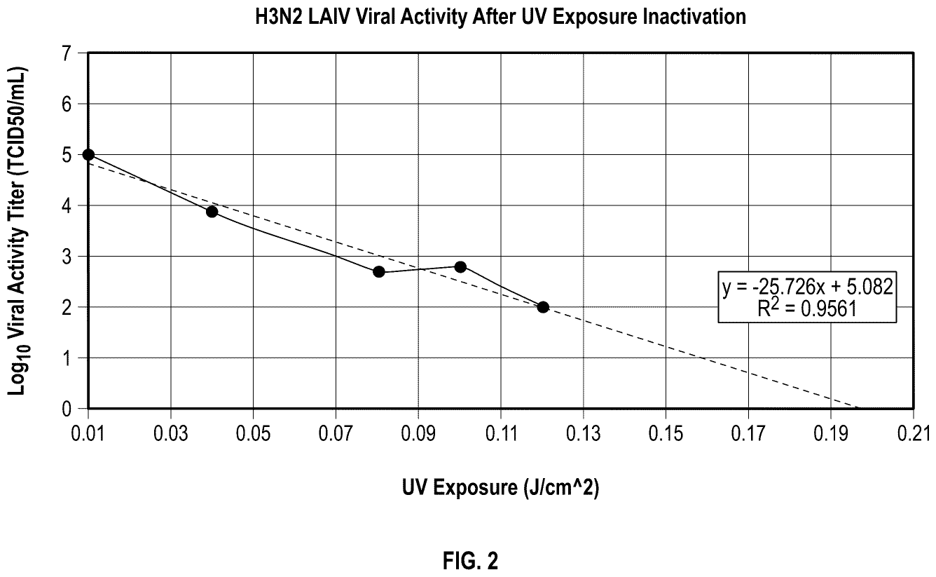

As it is seen from FIG. 2 approximately 0.2 J/cm² of UV irradiation is sufficient to fully inactivate 105 TCID50 of LAIV H3N2. The UVP Crosslinker can deliver irradiation doses of roughly 0.15 J/cm² per minute, allowing complete inactivation of high-titer viruses in a matter of minutes. UV irradiation of 0.16 J/cm² and higher dosage is below the limit of detection of the TCIDso assay. Full infectious virus inactivation at higher dosage was verified by multiple passages across larger cell cultures in flasks.

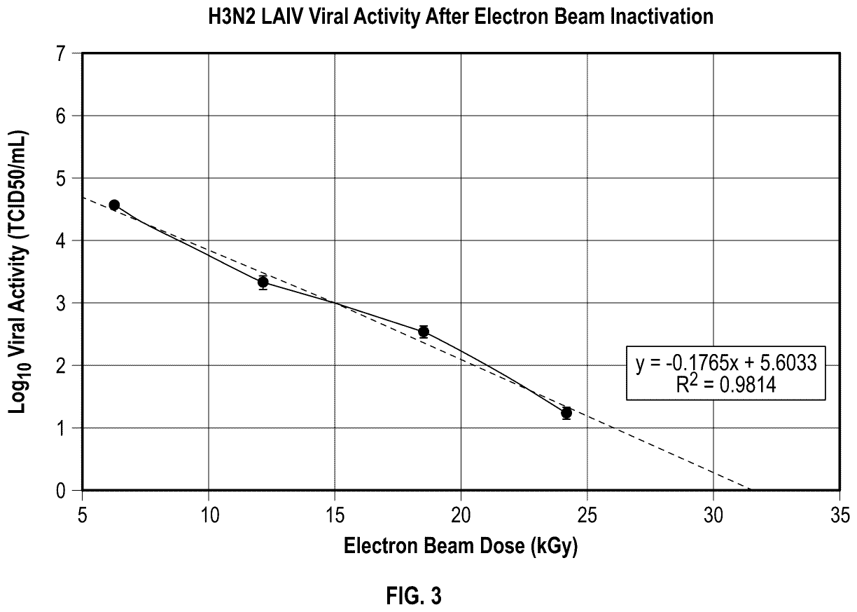

As it is seen from Table 2 no antigenicity damage was observed after applied UV radiation dose of below 1.8 J/cm² which is about 10 time bigger than the dose needed for more than million times of inactivation. This is surprising because in separate studies it was observed the beginning of antigenicity decrease after 48 kGy electron beam (EB) irradiation dose (see Table. 3). 48 kGy EB dose is only twice as high as the EB dose needed to decrease the viral activity about a million times (see FIG. 3). The data indicates that UVB inactivation is less damaging to viral epitopes than EB inactivation.

TABLE 2
H3N2 LAIV Antigenicity after UV Inactivation:

| | | | | HI titer |
|---|---|---|---|---|
| | HI titer | HI titer | HI titer | (geometric |
| H3N2 samples | #1 | #2 | #3 | mean) |
| Frozen control | 320 | 320 | 640 | 403.17 |
| PBV foam control | 320 | 320 | 320 | 320.00 |
| UV 0.04 J | 320 | 320 | 320 | 320.00 |
| UV 0.08 J | 320 | 320 | 320 | 320.00 |
| UV 0.10 J | 320 | 320 | 320 | 320.00 |
| UV 0.12 J | 320 | 320 | 320 | 320.00 |
| UV 0.16 J | 320 | 320 | 320 | 320.00 |
| UV 0.9 J | 320 | 320 | 320 | 320.00 |
| UV 1.8 J | 320 | 320 | 320 | 320.00 |

TABLE 3
H3N2 LAIV Antigenicity after EB Inactivation:

| | | | | HI titer |
|---|---|---|---|---|
| | HI titer | HI titer | HI titer | (geometric |
| H3N2 samples | #1 | #2 | #3 | mean) |
| Frozen control | 320 | 320 | 320 | 320.00 |
| PBV foam | 320 | 320 | 320 | 320.00 |
| control | | | | |
| EB 24 kGy | 320 | 320 | 320 | 320.00 |
| EB 48 kGy | 320 | 160 | 320 | 253.98 |

### Example 3. Inactivation of YF17D LAV Using UVA and UVB Irradiation

### Virus and Cell Lines:

Yellow fever vaccine strain YF-17D starter was propagated in Vero 76 cells (ATCC, CRL-1587). In brief, monolayers of Vero 76 cells were grown on T75 tissue culture flasks. Flasks were infected with YF-17D (original titer 3E6 PFU/mL) at a multiplicity of infection (MOI) of 0.01 and incubated at 37° C. in a 5% CO2 incubator for 5 days. Infected cell culture media was harvested and clarified by centrifugation for 10 minutes at 1300 rpm (Sorvall GLC-2B centrifuge). Virus titer was determined by standard plaque assay or focus-forming assay (described below) before and after inactivation.

### Yellow Fever Preservation Protocol:

YF-17D was mixed 1:1 with a preservation solution (PS) to form a preservation mixture (PM). The preservation solution comprised 25% sucrose, 8% sorbitol, 7% monosodium glutamate, 1% polyvinylpyrrolidone. The PM was divided into three batches PM1, PM2 and PM3. PM1 with the addition of psoralen (MilliporeSigma, P8399) to a final concentration of 10 μg/mL, PM2 with the addition of 4′-Aminomethyltrioxsalen hydrochloride (AMT) (MilliporeSigma, A4330) to a final concentration of 10 μg/mL, and PM3 without the addition of any psoralen compounds.

PMs were aliquoted in 5 ml serum vials, 0.5 ml of PM per vial (Wheaton borosilicate glass serum vials #223685). The vials were placed in a Virtis Genesis freeze-drier for stabilization using Preservation by Vaporization (PBV) technology (see U.S. Pat. No. 9,469,835). After drying, the vials were closed with rubber stoppers under vacuum and sealed.

### Ultraviolet Inactivation of YF-17D:

UV light transmission curve (FIG. 1) indicates roughly 90% transmission of 365 nm UV irradiation and roughly 80% transmission of 302 nm UV irradiation. A longwave UVP CL-1000L Ultraviolet Crosslinker (365 nm UV-A) and midwave UVP CL-1000M Ultraviolet Crosslinker (302 nm UV-B) (Analytik Jena, Germany) were used to irradiate vials. Sealed serum vials containing PBV-preserved YF-17D with and without psoralen compounds were subjected to a range of applied doses (0.05-1.0 J/cm²) of 365 nm or 302 nm ultraviolet irradiation to partially inactivate the yellow fever virus. Serum vials were placed upside down inside the crosslinker chamber to allow maximum surface area exposure to the UV light. The distance between the serum vials and UV lamps was about 11 cm. Samples were subsequently tested for viral infectivity via plaque assay or fluorescent focus-forming assay to evaluate the inactivation effect of UV-A irradiation (with or without psoralen compounds) and UV-B irradiation on preserved YF-17D virus.

### Plaque Assay:

Virus titer after UV-A irradiation was quantified by plaque assay. Following PBV drying and subsequent irradiation with 365 nm UV, preserved YF-17D was reconstituted with PBS containing 1% fetal bovine serum (FBS). 96-well tissue culture plates containing a confluent monolayer of Vero 76 cells were infected with 10-fold serial dilutions of YF-17D. After incubation for 1 h at 37° C., an overlay of carboxymethyl cellulose (CMC) and EMEM with 1% FBS was applied on top. The plates were incubated for 5 days at 37° C. with 5% CO₂, then cells were fixed with 4% paraformaldehyde and stained with crystal violet. Plaques were counted, and virus titer was calculated in PFU/mL.

### Fluorescent Focus-Forming Assay:

Virus titer after UV-A irradiation was quantified by focus-forming assay. Following PBV drying and subsequent irradiation with 365 nm UV, preserved YF-17D was reconstituted with PBS containing 1% fetal bovine serum (FBS). 96-well tissue culture plates containing a confluent monolayer of Vero 76 cells were infected with 10-fold serial dilutions of YF-17D. After incubation for 1 h at 37° C., an overlay of carboxymethyl cellulose (CMC) and EMEM with 1% FBS was applied on top. The plates were incubated for 5 days at 37° C. with 5% CO₂, then cells were fixed with a solution of 80% methanol and 20% acetone. Cells were stained intracellularly with YFV-specific mouse antibody (MilliporeSigma MAB984) for 1 h incubation at 37° C., followed by incubation for 1-2 hours with Alexa Fluor 488-conjugated goat anti-mouse IgG (ThermoFisher Scientific A31620). Foci were counted under a fluorescence microscope and virus titer was calculated in FFU/mL.

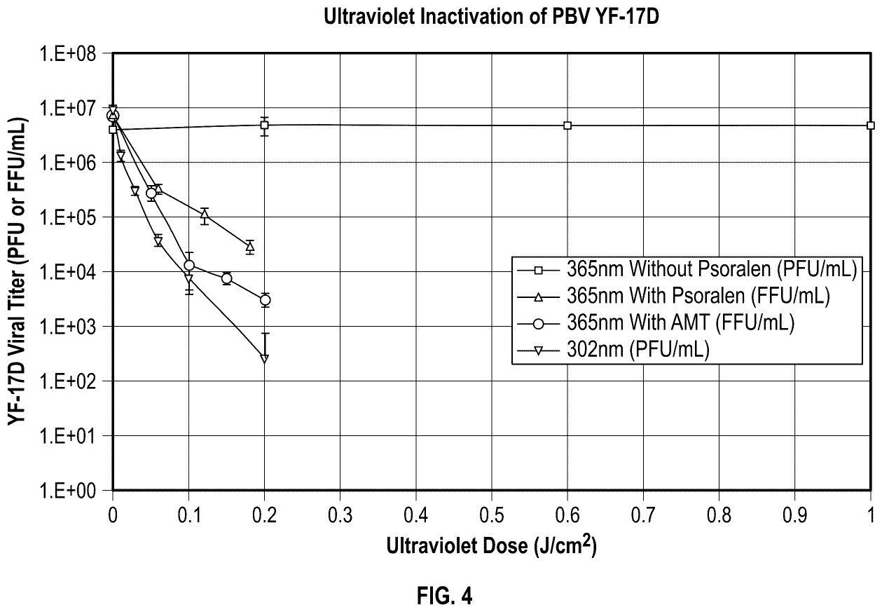

Results of viral activity measurements are shown in FIG. 4 and Table 4 below. The data indicates that for complete virus inactivation the applied dose should be about 1 J/cm².

TABLE 4
Viral activity measurements after UV inactivation of PBV YF-17D:

| UV 365 nm dose | | |
|---|---|---|
| (J/cm²) | PBV with psoralen (FFU/mL) | log loss |
| 0 | 6.80E+06 ± 0.35E+06 | N/A |
| 0.06 | 3.08E+05 ± 0.35E+05 | −1.34 |
| 0.12 | 1.08E+05 ± 0.36E+05 | −1.80 |
| 0.18 | 2.80E+04 ± 0.69E+04 | −2.39 |
| UV 365 nm dose | | |
| (J/cm²) | PBV with AMT (FFU/mL) | log loss |
| 0 | 6.92E+06 ± 0.99E+06 | N/A |
| 0.05 | 2.89E+05 ± 0.93E+05 | −1.38 |
| 0.10 | 1.33E+04 ± 0.87E+04 | −2.71 |
| 0.15 | 7.11E+03 ± 1.45E+03 | −2.99 |
| 0.20 | 3.11E+03 ± 0.93E+03 | −3.35 |

| UV 365 nm dose | PBV without psoralen compound | |
|---|---|---|
| (J/cm²) | (PFU/mL) | log loss |
| 0 | 4.00E+06 ± 2.18E+06 | N/A |
| 0.20 | 4.83E+06 ± 1.76E+06 | 0 |
| 0.60 | 4.67E+06 ± 0.76E+06 | 0 |
| 1.0 | 4.67E+06 ± 0.76E+06 | 0 |
| UV 302 nm dose | PBV without psoralen compound | |
| (J/cm²) | (PFU/mL) | log loss |
| 0 | 8.80E+06 ± 2.12E+06 | N/A |
| 0.01 | 1.36E+06 ± 0.35E+06 | −0.81 |
| 0.03 | 3.07E+05 ± 0.44E+05 | −1.46 |
| 0.06 | 3.73E+04 ± 0.78E+04 | −2.37 |
| 0.10 | 8.00E+03 ± 4.23E+03 | −3.04 |
| 0.20 | 2.67E+02 ± 4.62E+02 | −4.52 |

### Example 4. Validation of YF-17D Complete Inactivation

Inactivation of YF-17D virus after UV exposure was validated through inoculation of multiple passages of Vero 76 (ATCC, CRL-1587) cell monolayers to amplify any potential infectious units. In brief, Vero 76 cell monolayers were prepared in 25 cm² flasks, three flasks per test group including positive and negative control groups and inoculated with 5 mL of sample. Cells were cultured at 37° C. in 5% CO₂ incubators for 5 days and observed for cytopathic effect (CPE). Supernatant from each flask was inoculated onto new Vero 76 monolayers in 25 cm² flasks and incubated at 37° C. with 5% CO₂ for 5 days. Cells were observed for CPE, and supernatant from each flask was once more inoculated onto additional Vero 76 monolayers in 25 cm² flask. Cells were incubated at 37° C. with 5% CO₂ for 5 days, and observed for CPE.

Monolayers from this third passage were fixed and further verified via immunoperoxidase staining. Cells were fixed with 80% methanol and 20% acetone and stained intracellularly with YFV-specific mouse antibody (MilliporeSigma MAB984) for 2 h incubation at 37° C., followed by incubation for 2 h with HRP-conjugated goat anti-mouse IgG (Invitrogen G21040). Cells were visualized with an AEC staining kit (MilliporeSigma AEC101-1KT) and observed under microscope. We found that in the samples containing no psoralen irradiation with UVA with wavelength 365 nm did not inactivate YF-17D viruses at all at the irradiation dose 2.0 J/cm² or below. At the same time the irradiation at 1.5 J/sm2 or higher doses completely inactivated the viruses in the samples containing AMT (no CPE observed in any passage; no antigen detected in passage 3). However, after irradiation with 1 J/cm² we still observed CPE in the passages 2 and detected the antigen in the passage 3.

While various details, features, combinations are described in the illustrated embodiments, one having skill in the art will appreciate a myriad of possible alternative combinations and arrangements of the features disclosed herein. As such, the descriptions are intended to be enabling only, and non-limiting. Instead, the spirit and scope of the invention is set forth in the appended claims.

### INDUSTRIAL APPLICABILITY

The claimed invention is applicable to the pharmaceutical industry, specifically the vaccine industry though other portions of the pharmaceutical industry are also applicable.

### CITATION LIST

Allain J P, Hsu J, Pranmeth M, Hanson D, Stassinopoulos A, Fischetti L, Corash L, Lin L, 2006. Quantification of viral inactivation by photochemical treatment with amotosalen and UV A light, using a novel polymerase chain reaction inhibition method with preamplification. J Infect Dis 194: 1737-44.
Lin L, Cook D N, Wiesehahn G P, Alfonso R, Behrman B, Cimino G D, Corten L, Damonte P B, Dikeman R, Dupuis K, Fang Y M, Hanson C V, Hearst J E, Lin C Y, Londe H F, Metchette K, Nerio A T, Pu J T, Reames A A, Rheinschmidt M, Tessman J, Isaacs S T, Wollowitz S, Corash L, 1997. Photochemical inactivation of viruses and bacteria in platelet concentrates by use of a novel psoralen and long-wavelength ultraviolet light. Transfusion 37: 423-35.
Singh Y, Sawyer L S, Pinkoski L S, Dupuis K W, Hsu J C, Lin L, Corash L, 2006. Photochemical treatment of plasma with amotosalen and long-wavelength ultraviolet light inactivates pathogens while retaining coagulation function. Transfusion 46: 1168-77.
Nataraj A J, Black H S, Ananthaswamy H N, 1996. Signature p53 mutation at DNA cross-linking sites in 8-methoxypsoralen and ultraviolet A (PUVA)-induced murine skin cancers. Proc Natl Acadi Sci USA 93: 7961-5.
Rassner G, Steinert M, Bercher M, Rodermund O E, Henning D, Mey T, Heinzel M, 1987. [Chronic systemic toxicity of oral photochemotherapy using 8-methoxypsoralen and UVA]. Hautarzt 38: 10-7.
Maier H, Schemper M, Ortel B, Binder M, Tanew A, Honigsmann H, 1996. Skin tumors in photochemotherapy for psoriasis: a single-center follow-up of 496 patients. Dermatology 193: 185-91.
U.S. Pat. No. 4,124,598 issued Nov. 7, 1978.
U.S. Pat. No. 4,169,204 issued Sep. 25, 1979.
U.S. Pat. No. 4,196,281 issued Apr. 1, 1980.
U.S. Pat. No. 4,693,981 issued Sep. 15, 1987.
U.S. Pat. No. 6,455,286 issued Sep. 24, 2002.
U.S. Pat. No. 9,005,633 issued Apr. 14, 2015.
U.S. Pat. No. 9,469,835 issued Oct. 18, 2016.
U.S. Pat. No. 11,400,051 issued Aug. 2, 2022.

What is claimed is:

1. A method of producing a dry thermostable sterilized biopharmaceuticals, comprising:
stabilizing the biopharmaceutical at ambient temperatures using drying; and
subsequently exposing the stabilized biopharmaceutical to ultraviolet radiation at ambient temperatures having a dose between and inclusive of 0.2 J/cm² and 10 J/cm² to decrease viability of the microorganisms.

2. The method of claim 1, wherein the dry stabilized comprises two or more glass forming protective molecules comprising carbohydrates, sugar alcohols, amino acids, silica, derivatives thereof, polymers, or a combination thereof.

3. (canceled)

4. (canceled)

5. The method of claim 1, wherein the ultraviolet ionizing radiation dose comprises between and inclusive of 1 J/cm² and 10 J/cm².

6. The method of claim 1, wherein the biopharmaceutical comprises blood components, blood serum, stem cells, exosomes, growth factors and hormones, cytokines, secretome, antibodies, therapeutic proteins, postbiotics, inactivated microorganism, or a combination thereof.

7. The method of claim 1, wherein the ultraviolet ionizing radiation comprises a wavelength between and inclusive of 280 nm and 315 nm.

8. The method of claim 1, wherein the ultraviolet ionizing radiation comprises a wavelength between and inclusive of 315 nm and 400 nm.

9. The method of claim 8, further comprising adding psoralens and their derivatives like 4′-Aminomethyltrioxalen hydrochloride (AMT), 8-Methoxypsoralen (8-MOP), 4,5′,8-Trimethylpsoralen (TMP), or other molecules that interact with RNA or DNA under the UV irradiation.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The method of claim 1, wherein the dry thermostable sterilized biopharmaceutical comprises an inactivated vaccine.

24. The method of claim 23, further comprising: isolating nucleic acid aptamers that bind and are specific to the inactivated vaccines and other biopharmaceuticals in human serum.

25. (canceled)

26. (canceled)

27. (canceled)

28. (canceled)

29. (canceled)

30. (canceled)

31. (canceled)

32. (canceled)

33. (canceled)

34. A method of producing a dry thermostable sterilized biopharmaceutical using Preservation by Vaporization (PBV), comprising:
combining a microorganism suspension and a preservation solution to form a preservation mixture, the microorganism suspension comprising live virions or cellular microorganisms, the preservation solution comprising amino acids, one or more non-reducing disaccharides, and one or more monosaccharide derivatives and/or sugar alcohols;
drying the microorganism suspension by vaporization under vacuum to form a mechanically-stable glassy foam, said mechanically-stable glassy foam comprising:
less than five percent residual water content, and
a glass transition temperature greater than an ambient temperature at which the material will be stored,
wherein the live virions viruses or cellular microorganisms are immobilized in the mechanically-stable glassy foam; and
exposing the mechanically-stable glassy foam at ambient temperatures to an ultraviolet radiation having a wavelength from 280 to 400 nm and a dose between and inclusive of 0.2 J/cm² and 10 J/cm² to sterilize the biopharmaceutical.

35. The method of claim 34, wherein the microorganism suspension is preserved inside one or more serum vials.

36. The method of claim 34, wherein the preservation solution comprises one part by weight non-reducing monosaccharide derivatives and/or sugar alcohols and at least two parts by weight of sucrose, trehalose, isomalt or other non-reducing polysaccharides.

37. (canceled)

38. (canceled)

39. The method of claim 36, wherein said sugar alcohols are selected from the group consisting of: glycerol, sorbitol, mannitol, and erythritol.

40. The method of claim 34, the dry thermostable sterilized biopharmaceutical is stored at a temperature at or below 40° C., and the glass transition temperature is greater than or equal to 41° C.

41. (canceled)

42. (canceled)

43. The method of claim 34, wherein the cellular microorganisms comprise bacteria, fungi, vibrio, yeast, anthrax, listeria shigella, salmonella, E. coli, Yersinia pestis, cholera, or a combination thereof.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The method of claim 34, wherein the ultraviolet radiation comprises a wavelength between and inclusive of 290 nm and 310 nm.

58. (canceled)

59. The method of claim 34, further comprising adding psoralens to the microorganism suspension prior to drying.

60. (canceled)

61. (canceled)

62. (canceled)

63. (canceled)

64. (canceled)
